(19) United States
(12) Patent Application Publication
(10) Pub. No.: US 2023/0123395 A1
(43) Pub. Date: Apr. 20, 2023

(54) DOSAGE FORM COATING COMPOSITION AND METHOD OF MAKING AND USING THE SAME

(71) Applicant: SENSIENT COLORS LLC, St. Louis, MO (US)

(72) Inventors: Beverly A. Schad, Union, MO (US); Houston Smith, St. Louis, MO (US)

(21) Appl. No.: 17/876,348

(22) Filed: Jul. 28, 2022

Related U.S. Application Data
(63) Continuation of application No. 16/606,507, filed on Oct. 18, 2019, now abandoned.
(60) Provisional application No. 62/486,776, filed on Apr. 18, 2017.

(51) Int. Cl. A61K 9/28

(52) U.S. Cl. CPC A61K 9/2866; A61K 9/2813; A61K 9/282; A61K 9/286; A61K 9/288

(57) ABSTRACT

The present disclosure provides dosage form coating compositions and suspensions, coatings, coated dosage forms, and methods of making and using the same. The dosage form coating compositions can include a hydroxypropyl cellulose and a carboxymethylcellulose. The hydroxypropyl cellulose can be present in an amount by weight of at least 18.0% and at most 25.0%. The hydroxypropyl cellulose can be present in a ration of at least 1:1.4 and at most 1.4:1 relative to the carboxymethyl cellulose. The coated dosage forms can have an improved slip force, an improve swallowability as represented by an incline transit distance, or an improved gloss.

### CROSS-REFERENCE TO RELATED APPLICATIONS

This application claims the benefit of U.S. Provisional Application 62/486,776, filed Apr. 18, 2017, which is incorporated herein by reference in its entirety.

### BACKGROUND

Dosage form coating compositions are typically used to improve the properties of the dosage form to provide a user with an improved experience when taking the dosage form. Some key properties for coated dosage forms can include swallowability, hand feel, and appearance, among others. While dosage form coating compositions are known in the art, a need exists for dosage form coating compositions that can provide improved properties.

### BRIEF SUMMARY

In an aspect, the present disclosure provides a dosage form coating composition. The dosage form coating composition can include a hydroxypropyl cellulose and a carboxymethylcellulose. The hydroxypropyl cellulose can be present in an amount by weight of at least 18.0% and at most 25.0%. The carboxymethyl cellulose can be present an amount by weight of at least 18.0% and at most 25.0%. The dosage form coating composition, when applied to a 400 mg standard flat-faced radius-edged round table dosage form to a 3% weight gain, provides a coated dosage form having one or more of the following properties: a slip force, as measured by a texture analyzer, of less than 42.00 grams; an incline transit distance of at least 25 cm, as measured by placing the coated dosage form on an untreated stainless steel surface with a flat face of the coated dosage form contacting the untreated stainless steel surface, wetting the coated dosage form with 5 mL of de-ionized water to produce a wetted coated dosage form, raising the untreated stainless steel plate to an incline of 70 degrees relative to horizontal, and waiting for gravitational forces to act on the wetted coated dosage form thus causing the wetted coated dosage form to travel the incline transit distance, then measuring the incline transit distance; and a gloss of at least 18.0 gloss units.

In another aspect, the present disclosure provides a dosage form coating composition. The dosage form coating composition can include a hydroxypropyl cellulose and a carboxymethylcellulose. The hydroxypropyl cellulose can be present in a ration of at least 1:1.4 and at most 1.4:1 relative to the carboxymethyl cellulose. The dosage form coating composition, when applied to a 400 mg standard flat-faced radius-edged round table dosage form to a 3% weight gain, provides a coated dosage form having one or more of the following properties: a slip force, as measured by a texture analyzer, of less than 42.00 grams; an incline transit distance of at least 25 cm, as measured by placing the coated dosage form on an untreated stainless steel surface with a flat face of the coated dosage form contacting the untreated stainless steel surface, wetting the coated dosage form with 5 mL of de-ionized water to produce a wetted coated dosage form, raising the untreated stainless steel plate to an incline of 70 degrees relative to horizontal, and waiting for gravitational forces to act on the wetted coated dosage form thus causing the wetted coated dosage form to travel the incline transit distance, then measuring the incline transit distance; and a gloss of at least 18.0 gloss units.

In yet another aspect, the present disclosure provides a dosage form coating suspension. The dosage form coating suspension can include the dosage form coating composition as described herein and a solvent.

In a further aspect, the present disclosure provides a method of using the dosage form coating suspensions described herein. The method can include applying the dosage form coating suspension to an uncoated dosage form.

In another aspect, the present disclosure provides a method of making the dosage form coating compositions described herein. The method can include combining ingredients of the dosage form coating composition.

In yet another aspect, the present disclosure provides a coating. The coating can include non-volatile ingredients of the dosage form coating compositions described herein.

In a further aspect, the present disclosure provides a coated dosage form. The coated dosage form can include a dosage form and the coatings described herein.

### DETAILED DESCRIPTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise. It should be appreciated that aspects of the disclosure that are described with respect to a system are applicable to the methods, and vice versa, unless the context explicitly dictates otherwise.

Numeric ranges disclosed herein are inclusive of their endpoints. For example, a numeric range of between 1 and 10 includes the values 1 and 10. When a series of numeric ranges are disclosed for a given value, the present disclosure expressly contemplates ranges including all combinations of the upper and lower bounds of those ranges. For example, a numeric range of between 1 and 10 or between 2 and 9 is intended to include the numeric ranges of between 1 and 9 and between 2 and 10.

The present disclosure provides a dosage form coating composition. The dosage form coating composition can comprise a hydroxypropyl cellulose and a carboxymethyl cellulose.

In certain aspects, the hydroxypropyl cellulose can be Klucel EF (available commercially from Ashland Specialty Ingredients, Wilmington, Del.), Nisso HPC (available commercially from Nisso America Inc., New York, N.Y.), or the like.

The hydroxypropyl cellulose can be present in the dosage form coating composition in an amount by weight of at least 18.0%, at least 18.5%, at least 19.0%, at least 19.5%, at least 20.0%, at least 20.5%, at least 21.0%, at least 21.5%, or at least 22.0%. The hydroxypropyl cellulose can be present in the dosage form coating composition in an amount by weight of at most 25.0%, at most 24.5%, at most 24.0%, at most 23.5%, at most 23.0%, at most 22.5%, at most 22.0%, at most 21.5%, at most 21.0%, at most 20.5%, or at most 20.0%.

In certain aspects, the carboxymethyl cellulose can be sodium carboxymethyl cellulose, such as Blanose™ sodium carboxymethylcellulose (available commercially from Ashland Specialty Ingredients, Wilmington, Del.), WALOCEL™ (available commercially from The Dow Chemical Company, Midland, Mich.), or the like.

The carboxymethyl cellulose can be present in the dosage form coating composition in an amount by weight of at least 18.0%, at least 18.5%, at least 19.0%, at least 19.5%, at least 20.0%, at least 20.5%, at least 21.0%, at least 21.5%, or at least 22.0%. The carboxymethyl cellulose can be present in the dosage form coating composition in an amount by weight of at most 25.0%, at most 24.5%, at most 24.0%, at most 23.5%, at most 23.0%, at most 22.5%, at most 22.0%, at most 21.5%, at most 21.0%, at most 20.5%, or at most 20.0%.

The hydroxypropyl cellulose can be present in the dosage form coating composition in a ratio of at least 1:1.4, at least 1:1.3, at least 1:1.2, at least 1:1.1, or at least 1:1 relative to the carboxymethyl cellulose. The hydroxypropyl cellulose can be present in the dosage form coating composition in a ration of at most 1.4:1, at most 1.3:1, at most 1.2:1, at most 1.1:1, or at most 1:1 relative to the carboxymethyl cellulose.

The dosage form coating composition can, when applied to a placebo dosage form, including but not limited to, a standard convex round tablet dosage form, a compound cup round tablet dosage form, a flat-faced bevel-edged round tablet dosage form, a flat-faced radius-edged round tablet dosage form, or a standard convex capsule dosage form, to a 3% weight gain, provide a coated dosage form having one or more improved properties. The definitions of the various dosage form shapes can be found by referring to Tableting Specification Manual, 7th edition (Washington, D.C.: American Pharmacists Association, 2006), which is incorporated herein by reference in its entirety. As described elsewhere herein, the dosage form coating composition may be applied as a dosage form coating suspension.

In some aspects, the one or more improved properties can include an improved slip force. The slip force can be measured by a texture analyzer, such as the TA.XTPlus or TA.XTExpress texture analyzers, available commercially from Texture Technologies Corp., Hamilton, Mass. The slip force of the coated dosage form can be at most 42.00 grams, at most 41.00 grams, at most 40.00 grams, at most 39.50 grams, at most 39.00 grams, at most 38.50 grams, at most 38.00 grams, at most 37.50 grams, at most 37.00 grams, at most 36.50 grams, at most 36.00 grams, at most 35.50 grams, or at most 35.00 grams. The slip force of the coated dosage form can be at least 32.00 grams, at least 32.50 grams, at least 33.00 grams, at least 33.50 grams, at least 34.00 grams, at least 34.50 grams, or at least 35.00 grams.

In some aspects, the one or more improved properties can include an improved swallowability, as measured by an angular transit distance of a wetted coated dosage form along an untreated stainless steel plate oriented at an incline of 70 degrees (i.e., 70 degrees relative to horizontal and 20 degrees relative to vertical). The improved swallowability can be measured as follows: 1) the surface of the coated dosage form is wetted with ˜5-10 mL of de-ionized water while on the stainless steel plate; 2) the stainless steel plate is then raised to the 70-degree angle; 3) a period of time passes in order to allow the wetted coated dosage form to move a transit distance solely by the force of gravity; and 4) the transit distance of the wetted coated dosage form is measured. In certain cases this incline transit distance can be at least 25 cm, at least 30 cm, at least 35 cm, at least 40 cm, at least 45 cm, or at least 50 cm.

In some aspects, the one or more improved properties can be an improved gloss. The gloss of the coated dosage form can be at least 18.0 gloss units (gu), at least 19.0 gu, or at least 20.0 gu.

The dosage form coating composition can include an oil-based plasticizer. The oil-based plasticizer can be present in the dosage form coating composition in an amount by weight of at least 0.01%, at least 0.1%, at least 0.5%, at least 1.0%, at least 2.0%, at least 3.0%, at least 4.0%, at least 5.0%, at least 6.0%, at least 7.0%, or at least 8.0%. The oil-based plasticizer can be present in an amount by weight of at most 12.0%, at most 11.5%, at most 11.0%, at most 10.5%, at most 10.0%, at most 9.5%, at most 9.0%, at most 8.5%, or at most 8.0%.

The oil-based plasticizer can be selected from the group consisting of acetylated monoglycerides, medium chain triglycerides (MCT), propylene glycol dicaprylate/dicaprate (for example, Miglyol® 840, available commercially from Peter Cremer North America, Cincinnati, Ohio), oil based plasticizers contained within oil soluble flavor incorporations, and combinations thereof. In certain aspects, the oil-based plasticizer is acetylated monoglycerides.

The dosage form coating composition can include maltodextrin. The maltodextrin can be present in the dosage form coating composition in an amount by weight of at least 8.0%, at least 8.5%, at least 9.0%, at least 9.5%, or at least 10.0%. The maltodextrin can be present in the dosage form coating composition in an amount by weight of at most 25.0%, at most 20.0%, at most 17.5%, at most 15.0%, at most 12.5%, at most 12.0%, at most 11.0%, or at most 10.0%.

The dosage form coating composition can include talc. The talc can be present in the dosage form coating composition in an amount by weight of at least 0.01%, at least 0.1%, at least 0.5%, at least 1.0%, at least 2.5%, at least 5.0%, or at least 10.0%. The talc can be present in the dosage form coating composition in an amount by weight of at most 25.0%, at most 22.5%, at most 20.0%, at most 17.5%, at most 15.0%, at most 12.5%, or at most 10.0%.

The dosage form coating composition can include an opacifying agent. The opacifying agent can be present in the dosage form coating composition in an amount by weight of at least 10.0%, at least 10.5%, at least 11.0%, at least 11.5%, at least 12.0%, at least 12.5%, at least 13.0%, at least 13.5%, at least 14.0%, at least 14.5%, or at least 15.0%. The opacifying agent can be present in the dosage form coating composition in an amount by weight of at most 20.0%, at most 19.5%, at most 19.0%, at most 18.5%, at most 18.0%, at most 17.5%, at most 17.0%, at most 16.5%, at most 16.0%, at most 15.5%, or at most 15.0%.

The opacifying agent can be selected from the group consisting of titanium dioxide, calcium carbonate, Sensient® Avalanche™ (available commercially from Sensient Colors LLC, St. Louis, Mo.), other ingredients rendering opacification, and combinations thereof.

The dosage form coating composition can include a sweetening agent. The sweetening agent can be present in the dosage form coating composition in an amount by weight of at least 0.01%, at least 0.1%, at least 0.5%, at least 1.0%, at least 2.0%, at least 2.5%, at least 3.0%, at least 3.5%, at least 4.0%, at least 4.5%, at least 5.0%, at least 6.0%, at least 7.0%, or at least 8.0%. The sweetening agent can be present in the dosage form coating composition in an amount by weight of at most 10.0%, at most 9.5%, at most 9.0%, at most 8.5%, at most 8.0%, at most 7.5%, at most 7.0%, at most 6.0%, at most 5.0%, at most 4.0%, at most 3.0%, or at most 2.5%.

The sweetening agent can be selected from the group consisting of a sugar alcohol, an artificial sweetener, a natural sweetener, such as stevia, a sugar, and combinations thereof.

The sugar alcohol can be selected from the group consisting of sorbitol, mannitol, xylitol, isomalt, hydrogenated starch hydrolysates, and combinations thereof. The sugar alcohol can be present in the dosage form coating composition in an amount by weight of at least 0.01%, at least 0.1%, at least 0.5%, at least 1.0%, at least 2.0%, at least 2.5%, at least 3.0%, at least 3.5%, at least 4.0%, at least 4.5%, at least 5.0%, at least 5.5%, or at least 6.0%. The sugar alcohol can be present in the dosage form coating composition in an amount by weight of at most 8.0%, at most 7.5%, at most 7.0%, at most 6.5%, at most 6.0%, at most 5.5%, at most 5.0%, at most 4.5%, at most 4.0%, at most 3.5%, at most 3.0%, or at most 2.5%.

The artificial sweetener can be selected from the group consisting of sucralose, acesulfame, aspartame, and combinations thereof. The artificial sweetener can be present in the dosage form coating composition in an amount by weight of at least 0.01%, at least 0.1%, at least 0.5%, at least 1.0%, or at least 1.5%. The artificial sweetener can be present in the dosage form coating composition in an amount by weight of at most 2.0%, at most 1.5%, at most 1.0%, or at most 0.5%.

The sugar can be selected from the group consisting of sucrose, fructose, and combinations thereof.

The dosage form coating composition can include a flavorant or sensate. The flavorant can be a spray dried flavorant, a dried crystal flavorant, a granule flavorant, a liquid flavorant, or a combination thereof. The spray dried flavorant, the dried crystal flavorant, the granule flavorant, the liquid flavorant, or the combination thereof can comprise a synthetic flavoring agent, an artificial flavoring agent, a natural flavoring agent, or a combination thereof. The spray dried flavorant, the dried crystal flavorant, the granule flavorant, the liquid flavorant, or the combination thereof can provide a flavor selected from the group consisting of almond, amaretto, apple, green apple, apple-cherry-berry, apple-honey, apricot, bacon, banana, barbeque, beef, roast beef, beef steak, berry, berry blue, birch beer, spruce beer, blackberry, bloody mary, blueberry, boysenberry, brandy, bubble gum, butter, butter pecan, buttermilk, butterscotch, candy corn, cantaloupe, cantaloupe lime, caramel, carrot, cassia, caviar, celery, cereal, champagne, cherry, cherry cola, cherry maraschino, wild cherry, black cherry, red cherry, cherry-cola, chicken, chocolate, chocolate almond, cinnamon spice, citrus, citrus blend, citrus-strawberry, clam, cocoa, coconut, toasted coconut, coffee, coffee almond, cola, cola-vanilla, cookies & cream, cotton candy, cranberry, cranberry-raspberry, cream, cream soda, dairy type cream, creme de menthe, cucumber, black currant, dulce de leche, egg nog, pork fat, non-pork fat, anchovy fish, herring fish, sardine fish, frankfurter, fried garlic, sauteed garlic, gin, ginger ale, ginger beer, graham cracker type, grape, grape grapefruit, grapefruit-lemon, grapefruit-lime, grenadine, grill, guarana, guava, hazelnut, honey, roasted honey, ice cream cone, jalapeno, key lime, kiwi, kiwi-banana, kiwi-lemon-lime, kiwi-strawberry, kola champagne, lard type, lemon, lemon custard, lemonade, pink lemonade, lemon-lime, lime, malt, malted milk, mango, mango-pineapple, maple, margarita, marshmallow, meat type, condensed milk, cooked milk, mint, mirepoix, mocha, mochacinna, molasses, mushroom, sauteed mushroom, muskmelon, nectarine, neapolitan, green onion, sauteed onion, orange, orange cordial, orange creamsicle, orange creme, orange peach mango, orange strawberry banana, creamy orange, mandarin orange, orange-passion-guava, orange-pineapple, papaya, passion fruit, peach, peach-mango, peanut, roasted peanut, pear, pecan danish, pecan praline, pepper, peppermint, pimento, pina colada, pina colada/pineapple-coconut, pineapple, pineapple-orange, pistachio, pizza, pomegranate, baked potato, prune, punch, citrus punch, tropical punch, cherry fruit punch, grape punch, raspberry, black raspberry, blue raspberry, red raspberry, raspberry-blackberry, raspberry-ginger ale, raspberry-lime, root beer, rum, sangria, sarsaparilla, sassafras, sausage, sausage pizza, seafood, shrimp, hickory smoke, mesquite smoke, sour, sour cream, sour cream and onion, spearmint, strawberry, strawberry margarita, jam type strawberry, strawberry-kiwi, burnt sugar, tallow, tamarind, tangerine-lime, tangerine, tea, tequila, toffee, triple sec, tropical fruit mix, turkey, tutti frutti, vanilla, vanilla cream, vanilla custard, french vanilla, vegetable, vermouth, vinegar, balsamic vinegar, watermelon, whiskey, wildberry, wine, yogurt, and combinations thereof. The flavors described herein can be use alone or in combination with sensates described herein for experiential sensations of cooling, heating and tingling effects, such as use in combination with Sensient® Smoothenol® products.

The spray dried flavorant, the dried crystal flavorant, the granule flavorant, or a combination thereof can be present in the dosage form coating composition in an amount by weight of at least 0.1 at least 1.0% at least 2% at least 3.0%, at least 4.0%, at least 5.0%, at least 6.0%, at least 7.0%, at least 8.0%, at least 9.0%, at least 10.0%, at least 11.0%, at least 12.0%, or at least 12.5%. The spray dried flavorant, the dried crystal flavorant, the granule flavorant, or a combination thereof can be present in the dosage form coating composition in an amount by weight of at most 15.0%, at most 14.0%, at most 13.0%, at most 12.0%, at most 11.0%, at most 10.0%, at most 9.0%, at most 8.0%, at most 7.0%, at most 6.0%, or at most 5.0%.

The liquid flavorant can be present in the dosage form coating composition in an amount by weight of at least 0.1%, at least 0.5%, at least 1.0%, at least 2.0%, at least 2.5%, at least 3.0%, at least 3.5%, at least 4.0%, at least 4.5%, at least 5.0%, at least 6.0%, at least 7.0%, or at least 8.0%. The liquid flavorant can be present in the dosage form coating composition in an amount by weight of at most 9.0%, at most 8.0%, at most 7.0%, at most 6.0%, at most 5.0%, at most 4.0%, at most 3.0%, at most 2.0%, or at most 1.0%.

The dosage form coating composition can include a sensate. The sensate can be a spray dried sensate, a dried crystal sensate, a granule sensate, a liquid sensate, or a combination thereof. The spray dried sensate, the dried crystal sensate, the granule sensate, the liquid sensate, or a combination thereof can provide a hot sensation, a cool sensation, a tingling sensation, or a combination thereof. In some cases, the sensate can be combined with a flavorant to provide a combination flavorant and sensate that combined the flavors and the sensations disclosed herein. In the event that a flavorant is combined with a sensate, the combination flavorant and sensate should be present in an amount that is equal to the amounts described herein with respect to flavorants and sensates.

The spray dried sensate, the dried crystal sensate, the granule sensate, or a combination thereof can be present in the dosage form coating composition in an amount by weight of at least 1% at least 2% at least 3.0%, at least 4.0%, at least 5.0%, at least 6.0%, at least 7.0%, at least 8.0%, at least 9.0%, at least 10.0%, at least 11.0%, at least 12.0%, or at least 12.5%. The spray dried sensate, the dried crystal sensate, the granule sensate, or a combination thereof can be present in the dosage form coating composition in an amount by weight of at most 15.0%, at most 14.0%, at most 13.0%, at most 12.0%, at most 11.0%, at most 10.0%, at most 9.0%, at most 8.0%, at most 7.0%, at most 6.0%, or at most 5.0%.

The liquid sensate can be present in the dosage form coating composition in an amount by weight of at least 0.1%, at least 0.5%, at least 1.0%, at least 2.0%, at least 2.5%, at least 3.0%, at least 3.5%, at least 4.0%, at least 4.5%, at least 5.0%, at least 6.0%, at least 7.0%, or at least 8.0%. The liquid sensate can be present in the dosage form coating composition in an amount by weight of at most 9.0%, at most 8.0%, at most 7.0%, at most 6.0%, at most 5.0%, at most 4.0%, at most 3.0%, at most 2.0%, or at most 1.0%.

The dosage form coating composition can include a flavor masking agent. The flavor masking agent can be selected from the group consisting of Smoothenol®, Smoothenol 2G® or numerical G smoothenol; such as 3G, 4G and in the forms of BitterFix™, AstringentFix™ FunctionalFix™, BurnFix™, SourFix™ (all available commercially from Sensient Flavors LLC, Hoffman Estates, Ill.), and combinations thereof. The flavor masking agent can be present in an amount by weight of at least 0.1%, at least 0.5%, at least 1.0%, at least 2.0%, at least 2.5%, at least 3.0%, at least 3.5%, at least 4.0%, at least 4.5%, at least 5.0%, at least 6.0%, at least 7.0%, or at least 8.0%. The flavor masking gent can be present in an amount by weight of at most 9.0%, at most 8.0%, at most 7.0%, at most 6.0%, at most 5.0%, at most 4.0%, at most 3.0%, at most 2.0%, or at most 1.0%. In some cases, the flavor masking agent can be combined with a flavorant, a sweetener, a sweetener enhancer, or the like. In some cases, the flavor masking agent can be contained in a combination product, such as Mafco's Magnasweet® line of products (available commercially from MAFCO Worldwide LLC, Camden, N.J.).

The dosage form coating composition can include a colorant. The colorant can be selected from the group consisting of a pigment, a dye, an exempt colorant (i.e., a colorant from a natural source), and combinations thereof. The colorant can be present in an amount by weight of at least 0.01% at least 0.1%, at least 0.5%, at least 1.0%, at least 2.0%, at least 2.5%, at least 3.0%, at least 3.5%, at least 4.0%, at least 4.5%, at least 5.0%, at least 6.0%, at least 7.0%, or at least 8.0%. The colorant can be present in an amount by weight of at most 20.0%, at most 17.5%, at most 15.0%, at most 12.5%, or at most 10.0%.

The dosage form coating composition can include an acidifying agent. The acidifying agent can be selected from the group consisting of citric acid, malic acid, ascorbic acid, and combinations thereof. The acidifying agent can be present in the dosage form coating composition in an amount by weight of at least 0.01%, at least 0.1%, at least 0.25%, or at least 0.5%. The acidifying agent can be present in the dosage form coating composition in an amount by weight of at most 1.0%, at most 0.9%, at most 0.75%, or at most 0.5%.

The dosage form coating composition can be substantially free of various components that are commonly used in the dosage form coating arts. The dosage form coating composition can be substantially free of hydroxypropyl methyl cellulose. The dosage form coating composition can be substantially free of polyethylene glycol. The dosage form coating composition can be substantially free of polyvinyl alcohol. The dosage form coating composition can be substantially free of povidone.

The present disclosure provides a dosage form coating suspension. The dosage form coating suspension can include the dosage form coating composition, as described elsewhere herein, and a solvent. The solvent can be selected from the group consisting of water, alcohol, such as methanol, ethanol, isopropanol, butyl alcohol, and combinations thereof.

The dosage form coating suspension can have a solids content of at least 8.0%, at least 8.5%, at least 9.0%, at least 9.5%, at least 10.0%, at least 10.5%, or at least 11.0%. The dosage form coating suspension can have a solids content of at most 13.0%, at most 12.5%, at most 12.0%, at most 11.5%, at most 11.0%, at most 10.5%, or at most 10.0%.

The present disclosure provides a coating. The coating is the result of applying the dosage form coating suspension to an article in accordance with the methods described herein. The coating can include the same or substantially similar components as described elsewhere herein with respect to the dosage form coating composition, minus any volatile components that are removed in the coating process, as would be understood by a person having ordinary skill in the art.

The present disclosure provides a coated dosage form. The coated dosage form is the result of applying the dosage form coating suspension to a dosage form in accordance with the methods described herein. The coated dosage form includes the dosage form and the coating, as described elsewhere herein. While the properties of the dosage form coating composition are described with respect to the coating of a specific dosage form in a specific fashion (as described herein), the composition can be used to coat a wide variety of dosage forms, including but not limited to, tablets, caplets, capsules, softgels, dissolvable strips, multiparticulates, and the like.

The present disclosure provides a method of making a dosage form coating composition and/or suspension.

The method of making the dosage form composition can include combining and/or milling the various components of the dosage form coating composition.

The method of making the dosage form coating suspension can include: 1) stirring a desired amount of solvent at a level sufficient to generate a vortex; 2) adding a desired amount of the dosage form coating composition; and 3) mixing until a suspension forms.

The present disclosure provides a method of using a dosage form coating composition and/or suspension.

In cases where the dosage form coating composition is the starting material, the method of using the dosage form coating composition can include preparing a dosage form coating suspension having a solids content as described elsewhere herein. The method can then continue with the method described below with respect to the dosage form coating suspension.

In cases where the dosage form coating suspension is the starting material, the method of using the dosage form coating suspension can include applying the dosage form coating suspension to a plurality of uncoated dosage forms. The applying can be in compliance with the parameters outlined in Table 1 below.

TABLE 1

| Pan size | 12 in | 15 in | 19 in | 24 in | 30 in | 36 in | 48 in | 60 in | 66 in |
|---|---|---|---|---|---|---|---|---|---|
| Air Volume | 130 to 200 | 250 to 300 | 275 to 350 | 275 to 400 | 400 to 500 | 700 to 1200 | 1500 to 2000 | 3800 to 4800 | 4000 to 6000 |
| cfm | | | | | | | | | |
| Inlet Temp | 50 to 65 | 50 to 65 | 55 to 65 | 60 to 65 | 62 to 65 | 62 to 65 | 65 to 75 | 65 to 75 | 65 to 75 |
| (° C.) | | | | | | | | | |
| Outlet Temp | 42 to 50 | 42 to 50 | 42 to 50 | 42 to 50 | 45 to 50 | 45 to 50 | 45 to 50 | 45 to 50 | 45 to 50 |
| (° C.) | | | | | | | | | |
| Bed Temp | 40 to 47 | 40 to 47 | 40 to 47 | 40 to 47 | 40 to 47 | 40 to 47 | 40 to 47 | 40 to 47 | 40 to 47 |
| (° C.) | | | | | | | | | |
| Pre-Warm | 40 to 45 | 40 to 45 | 40 to 45 | 40 to 45 | 40 to 45 | 40 to 45 | 40 to 45 | 40 to 45 | 40 to 45 |
| Temp (° C.) | | | | | | | | | |
| Spray Rate | 10 | 10 to 20 | 35 to 50 | 40 to 70 | 150 to 175 | 175 to 225 | 250 to 400 | 800 to 1000 | 900 to 1000 |
| g/min | | | | | | | | | |
| Atomizing | 15 | 20 | 30 | 30 | 40 | 50 | 50 to 70 | 50 to 70 | 50 to 70 |
| Air (psi) | | | | | | | | | |
| Pattern Air (psi) | 20 | 25 | 35 | 35 | 45 | 55 | 50 to 75 | 50 to 75 | 50 to 75 |
| Number of Guns | 1 | 1 | 1 to 2 | 2 | 2 | 2 to 3 | 3 to 4 | 4 to 6 | 6 |
| Pan Speed (rpm) | 20 to 25 | 15 to 20 | 15 to 18 | 12 to 15 | 10 to 12 | 7 to 11 | 5 to 8 | 4 to 6 | 2 to 4 |
| Charge wt (kg) | 1 | 1 to 3 | 3 to 9 | 15 to 20 | 40 to 50 | 60 to 75 | 120 to 200 | 250 to 350 | 450 to 600 |

### EXAMPLES

### Example 1

| Ingredient | % |
|---|---|
| Klucel EF | 21.000% |
| Na CMC | 21.000% |
| Maltodextrin | 10.000% |
| Talc | 8.200% |
| Sorbitol | 6.000% |
| Sucralose | 1.000% |
| Acetylated Monoglycerides | 8.000% |
| Titanium Dioxide | 15.000% |
| Coolenol ® (available commercially from Sensient | 3.000% |
| Flavors LLC, St. Louis, MO) | |
| Vanilla | 6.500% |
| Citric Acid | 0.300% |

### Example 2

| Ingredient | % |
|---|---|
| Klucel EF | 21.000% |
| Na CMC | 21.000% |
| Maltodextrin | 10.000% |
| Talc | 18.000% |
| Sorbitol | 6.000% |
| Sucralose | 1.000% |
| Acetylated Monoglycerides | 8.000% |
| Titanium Dioxide | 15.000% |

### Example 3

| Ingredient | % |
|---|---|
| HPC | 21.000% |
| Na CMC | 21.000% |
| Maltodextrin | 10.000% |
| Talc | 10.000% |
| Sorbitol | 5.000% |
| Acetylated Monoglycerides | 8.000% |
| Stevia | 1.000% |
| Sensipearl ™ Blue (available commercially from | 12.000% |
| Sensient Colors LLC, St. Louis, MO) | |
| Spirulina (microfine blue SE) | 3.000% |
| Peppermint oil-soluble | 7.000% |
| Coolenol ® oil-soluble | 2.000% |

### Example 4

Two 600 mg standard caplet cores were coated to a 3% weight gain, one with the composition described above in Example 2 and the other with hydroxypropylmethylcellulose (HPMC). The two coated caplets were subjected to the swallowability test described above. The caplet coated with the composition of Example 2 achieved a transit distance of greater than 25 cm, whereas the caplet coated with HPMC did not move. Thus, the composition of Example 2 exhibited superior swallowability, particularly when compared with HPMC.

The particular aspects disclosed above are illustrative only, as the technology may be modified and practiced in different but equivalent manners apparent to those skilled in the art having the benefit of the teachings herein. Furthermore, no limitations are intended to the details of construction or design herein shown, other than as described in the claims below. It is therefore evident that the particular aspects disclosed above may be altered or modified and all such variations are considered within the scope and spirit of the technology. Accordingly, the protection sought herein is as set forth in the claims below.

1. A dosage form coating composition comprising:
a hydroxypropyl cellulose in an amount by weight of at least 18.0% and at most 25.0%; and
a carboxymethyl cellulose in an amount by weight of at least 18.0% and at most 25.0%,
wherein the dosage form coating composition, when applied to a 400 mg standard flat-faced radius-edged round tablet dosage form to a 3% weight gain, provides a coated dosage form having one or more of the following properties:
a slip force, as measured by a texture analyzer, of less than 42.00 grams;
an incline transit distance of at least 25 cm, as measured by placing the coated dosage form on an untreated stainless steel surface with a flat face of the coated dosage form contacting the untreated stainless steel surface, wetting the coated dosage form with 5 mL of de-ionized water to produce a wetted coated dosage form, raising the untreated stainless steel plate to an incline of 70 degrees relative to horizontal, and waiting for gravitational forces to act on the wetted coated dosage form thus causing the wetted coated dosage form to travel the incline transit distance, then measuring the incline transit distance; and
a gloss of at least 18.0 gloss units.

2. A dosage form coating composition comprising:
a hydroxypropyl cellulose; and
a carboxymethyl cellulose,
wherein the hydroxypropyl cellulose is present in a ratio of at least 1:1.4 and at most 1.4:1 relative to the carboxymethyl cellulose, and
wherein the dosage form coating composition, when applied to a 400 mg standard flat-faced radius-edged round tablet dosage form to a 3% weight gain, provides a coated dosage form having one or more of the following properties:
a slip force, as measured by a texture analyzer, of less than 42.00 grams;
an incline transit distance of at least 25 cm, as measured by placing the coated dosage form on an untreated stainless steel surface with a flat face of the coated dosage form contacting the untreated stainless steel surface, wetting the coated dosage form with 5 mL of de-ionized water to produce a wetted coated dosage form, raising the untreated stainless steel plate to an incline of 70 degrees relative to horizontal, and waiting for gravitational forces to act on the wetted coated dosage form thus causing the wetted coated dosage form to travel the incline transit distance, then measuring the incline transit distance; and
a gloss of at least 18.0 gloss units.

3. The dosage form coating composition of claim 2, wherein the hydroxypropyl cellulose and the carboxymethyl cellulose are present in a combined amount by weight of the dosage form coating composition of at least 36.0% and at most 50.0%.

4. The dosage form coating composition of claim 1, the coated dosage form having the slip force, as measured by a texture analyzer, of less than 42.00 grams.

5. The dosage form coating composition of claim 1, the coated dosage form having the incline transit distance of at least 25 cm, as measured by placing the coated dosage form on the untreated stainless steel surface with the convex face of the coated dosage form contacting the untreated stainless steel surface, wetting the coated dosage form with 5 mL of de-ionized water to produce the wetted coated dosage form, raising the untreated stainless steel plate to an incline of 70 degrees relative to horizontal, and waiting for gravitational forces to act on the wetted coated dosage form thus causing the wetted coated dosage form to travel the incline transit distance, then measuring the incline transit distance.

6. The dosage form coating composition of claim 1, the coated dosage form having the gloss of at least 18.0 gloss units.

7. The dosage form coating composition of claim 1, the dosage form coating composition further comprising an oil-based plasticizer in an amount by weight of at least 0.01% and at most 12.0%.

8. The dosage form coating composition of claim 7, wherein the oil-based plasticizer comprises acetylated monoglycerides.

9. The dosage form coating composition of claim 1, the dosage form coating composition further comprising maltodextrin in an amount by weight of at least 8.0% and at most 25.0%.

10. The dosage form coating composition of claim 1, the dosage form coating composition further comprising talc in an amount by weight of at least 0.01% and at most 25.0%.

11. The dosage form coating composition of claim 2, the coated dosage form having the slip force, as measured by a texture analyzer, of less than 42.00 grams.

12. The dosage form coating composition of claim 2, the coated dosage form having the incline transit distance of at least 25 cm, as measured by placing the coated dosage form on the untreated stainless steel surface with the convex face of the coated dosage form contacting the untreated stainless steel surface, wetting the coated dosage form with 5 mL of de-ionized water to produce the wetted coated dosage form, raising the untreated stainless steel plate to an incline of 70 degrees relative to horizontal, and waiting for gravitational forces to act on the wetted coated dosage form thus causing the wetted coated dosage form to travel the incline transit distance, then measuring the incline transit distance.

13. The dosage form coating composition of claim 2, the coated dosage form having the gloss of at least 18.0 gloss units.

14. The dosage form coating composition of claim 2, the dosage form coating composition further comprising an oil-based plasticizer in an amount by weight of at least 0.01% and at most 12.0%.

15. The dosage form coating composition of claim 14, wherein the oil-based plasticizer comprises acetylated monoglycerides.

16. The dosage form coating composition of claim 2, the dosage form coating composition further comprising maltodextrin in an amount by weight of at least 8.0% and at most 25.0%.

17. The dosage form coating composition of claim 2, the dosage form coating composition further comprising talc in an amount by weight of at least 0.01% and at most 25.0%.

18-39. (canceled)

40. A dosage form coating suspension comprising the dosage form coating composition of claim 1 and a solvent.

41. The dosage form coating suspension of claim 40, wherein the dosage form coating suspension has a solids content of at least 8.0% and at most 13.0%.

42-51. (canceled)

52. A dosage form coating suspension comprising the dosage form coating composition of claim 2 and a solvent.
